(19) United States
(12) Patent Application Publication
(10) Pub. No.: US 2026/0021037 A1
(43) Pub. Date: Jan. 22, 2026

(54) Time-Release Vaginal Lubrication Capsule Device

(71) Applicant: Leon Boatwright, Clarks Hill, SC (US)

(72) Inventor: Leon Boatwright, Clarks Hill, SC (US)

(21) Appl. No.: 18/952,279

(22) Filed: Nov. 19, 2024

Related U.S. Application Data
(60) Provisional application No. 63/672,806, filed on Jul. 18, 2024.

(51) Int. Cl. A61K 9/00

(52) U.S. Cl. CPC A61K 9/0036

(57) ABSTRACT

A time-release vaginal lubrication capsule device is provided. The device is comprised of a capsule designed to release lubricant gradually when exposed to body heat. The capsule, made from biodegradable polymers such as poly (lactic-co-glycolic acid) (PLGA), dissolves at a controlled rate, releasing the lubricant stored within. The dissolution rate can be adjusted by varying the thickness or using multi-layer designs for phased release. Lubricant options include water-based formulas, some with micro-encapsulated beads for extended-release. Additional features include the incorporation of soothing agents, flavors, and scents, as well as a syringe for insertion and a sterilization cylinder for cleaning.

### CROSS-REFERENCE TO RELATED APPLICATION

The present application claims priority to, and the benefit of, U.S. Provisional Application No. 63/672,806, which was filed on Jul. 18, 2024, and is incorporated herein by reference in its entirety.

### FIELD OF THE INVENTION

The present invention relates generally to the field of vaginal lubricants. More specifically, the present invention relates to a dissolving capsule containing a lubricant for time-released vaginal lubrication. Accordingly, the present disclosure makes specific reference thereto. Nonetheless, it is to be appreciated that aspects of the present invention are also equally applicable to other like applications, devices, and methods of manufacture.

### BACKGROUND

For women experiencing menopause or for others dealing with vaginal dryness due to various factors, finding a suitable, effective lubricant can often be a daunting task. Many commonly available lubricants require application immediately before intimacy, which can interrupt the flow of the moment, leading to awkwardness or discomfort. This last-minute application process may feel rushed or inconvenient, especially in situations where spontaneity is valued. Moreover, many lubricants on the market are messy, difficult to manage, and require direct application with the hands, which may not always be desirable or comfortable for everyone.

In addition to these challenges, certain lubricants may leave behind residues or stickiness, further complicating their use and potentially detracting from the overall experience. For women dealing with hormonal changes, such as those going through menopause, or for individuals with medical conditions that contribute to dryness, the search for a product that not only provides long-lasting relief but also eliminates the need for immediate or frequent reapplication can feel overwhelming.

Therefore, there exists a long-felt need in the art for an improved lubricant. There also exists a long-felt need in the art for a time-release vaginal lubrication capsule device. More specifically, there exists a long-felt need in the art for a time-release vaginal lubrication capsule device that provides an improved vaginal lubricant that does not require direct application by the user. In addition, there exists a long-felt need in the art for a time-release vaginal lubrication capsule device that provides an improved vaginal lubricant that does not require direct application by the user and wherein the device can release lubricant over time. Further, there exists a long-felt need in the art for a time-release vaginal lubrication capsule device that provides an improved vaginal lubricant that does not require direct application by the user, can release lubricant over time, and is not messy.

The subject matter disclosed and claimed herein, in one embodiment thereof, comprises a time-release vaginal lubrication capsule device. The device is comprised of a capsule designed to release lubricant gradually when exposed to body heat. The capsule, made from biodegradable polymers such as poly (lactic-co-glycolic acid) (PLGA), dissolves at a controlled rate, releasing the lubricant stored within. The dissolution rate can be adjusted by varying the thickness or using multi-layer designs for phased release. Lubricant options include water-based formulas, some with micro-encapsulated beads for extended-release. Additional features include the incorporation of soothing agents, flavors, and scents, as well as a syringe for insertion and a sterilization cylinder for cleaning.

In this manner, the time-release vaginal lubrication capsule device of the present invention accomplishes all the forgoing objectives and provides an improved vaginal lubricant. More specifically, the device provides an improved vaginal lubricant that does not require direct application of the lubricant by the user. In addition, the device can release lubricant over time. Further, the device does not provide a messy user experience.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a time-release vaginal lubrication capsule device. The device is comprised of a capsule. During use, the capsule is inserted into a user's vagina, where it reacts to the body's natural heat. More specifically, the capsule is made of a biocompatible, heat-sensitive, and dissolvable polymer. Upon reaching a heating threshold within the vagina, the polymer dissolves in a controlled manner, allowing the release of a lubricant stored within the capsule. This ensures that the lubricant is released slowly and steadily into the vaginal canal over a designated period of time, providing sustained moisture and comfort during intimate activities.

The rate at which the capsule dissolves can be adjusted depending on the desired time-release duration. For example, in embodiments where a longer release period is needed, the capsule wall may be made thicker. A thicker wall slows the dissolution process, delaying the full release of the lubricant. Conversely, for shorter time intervals, a thinner wall may be used, allowing quicker breakdown and more rapid release of the lubricant. In another embodiment, the capsule may be constructed with a multi-layer design. The capsule would feature a plurality of wall layers that dissolve sequentially. Lubricant is positioned between each layer, ensuring that with the dissolution of each layer, a fresh batch of lubricant is released. This tiered release system allows the device to dispense lubricant at predetermined intervals, ensuring ongoing moisture and lubrication for the user over an extended period.

In one embodiment, the lubricant contained within the capsule is in liquid form, designed to be quickly absorbed by the vaginal tissue for immediate relief from dryness. In another embodiment, the lubricant may be micro-encapsulated in beads. These beads, like the capsule itself, are made from a dissolvable material, often similar or identical to the biodegradable polymer used in the capsule. The beads dissolve over time at varying rates, creating a staggered release of the lubricant.

The lubricant itself is preferably water-based, which allows for easy absorption and reduces the risk of irritation or allergic reactions compared to oil-based products. The lubricant may also contain soothing agents such as hyaluronic acid and/or aloe vera to promote healing and hydration, particularly for users who experience discomfort due to dryness or irritation. These agents are known for their ability to enhance moisture retention and promote healthy skin or tissue recovery, making the lubricant more than just a short-term solution for dryness, but also a tool for improving overall vaginal health. In some embodiments, the lubricant may also be flavored and/or scented to enhance the user experience.

Accordingly, the time-release vaginal lubrication capsule device of the present invention is particularly advantageous as it provides an improved vaginal lubricant that does not require direct application of the lubricant by the user. In addition, the device can release lubricant over time. Further, the device does not provide a messy user experience. In this manner, the time-release vaginal lubrication capsule device overcomes the limitations of existing vaginal lubricants known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a time-release vaginal lubrication capsule device of the present invention in accordance with the disclosed architecture;

FIG. 2 illustrates a side cross-sectional view of one potential embodiment of a time-release vaginal lubrication capsule device of the present invention in accordance with the disclosed architecture; and

FIG. 3 illustrates a front cross-sectional view of one potential embodiment of a time-release vaginal lubrication capsule device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved lubricant. There also exists a long-felt need in the art for a time-release vaginal lubrication capsule device. More specifically, there exists a long-felt need in the art for a time-release vaginal lubrication capsule device that provides an improved vaginal lubricant that does not require direct application by the user. In addition, there exists a long-felt need in the art for a time-release vaginal lubrication capsule device that provides an improved vaginal lubricant that does not require direct application by the user and wherein the device can release lubricant over time. Further, there exists a long-felt need in the art for a time-release vaginal lubrication capsule device that provides an improved vaginal lubricant that does not require direct application by the user, can release lubricant over time, and is not messy.

The present invention, in one exemplary embodiment, is comprised of a time-release vaginal lubrication capsule device. The device is comprised of a capsule that is inserted into the user's vagina, where it responds to the body's natural heat. Specifically, the capsule is made of a biocompatible, heat-sensitive, and dissolvable polymer. Upon reaching a certain temperature inside the vagina, the polymer dissolves in a controlled manner, gradually releasing the lubricant contained within. This process ensures that the lubricant is slowly and steadily dispensed into the vaginal canal over a designated period, providing ongoing moisture and comfort during intimate activities.

The dissolution rate of the capsule can be adjusted according to the desired release time. For instance, if a longer release period is preferred, the capsule wall can be made thicker, which slows down the dissolution process and delays the release of the lubricant. Conversely, for a quicker release, a thinner wall may be used, allowing the lubricant to be released more rapidly. In another embodiment, the capsule may feature a multi-layered design, with each layer dissolving sequentially. Lubricant is placed between these layers, ensuring that with the dissolution of each layer, a new portion of lubricant is released. This tiered release system allows the device to provide moisture at regular intervals, ensuring continuous lubrication over an extended period.

In one embodiment, the lubricant inside the capsule is in liquid form and is designed to be quickly absorbed by the vaginal tissue, offering immediate relief from dryness. In another embodiment, the lubricant may be micro-encapsulated in beads. These beads, like the capsule itself, are made from a dissolvable material, often similar to the biodegradable polymer used in the capsule. The beads dissolve at varying rates, creating a staggered release of the lubricant.

The lubricant is preferably water-based, which promotes easy absorption and reduces the likelihood of irritation or allergic reactions compared to oil-based alternatives. The lubricant may also include soothing agents such as hyaluronic acid and/or aloe vera, which support healing and hydration, particularly for users who experience discomfort from dryness or irritation. These agents are known for their ability to enhance moisture retention and promote healthy tissue, making the lubricant not just a short-term solution for dryness but a means to improve overall vaginal health. In some embodiments, the lubricant may also be flavored or scented to enhance the user's experience.

Accordingly, the time-release vaginal lubrication capsule device of the present invention is particularly beneficial as it provides a more convenient solution for vaginal lubrication, eliminating the need for the user to apply the lubricant directly. Moreover, the device is designed to release lubricant over time, offering a cleaner, mess-free experience. In this way, the time-release vaginal lubrication capsule device addresses and resolves many of the limitations found in traditional vaginal lubricants.

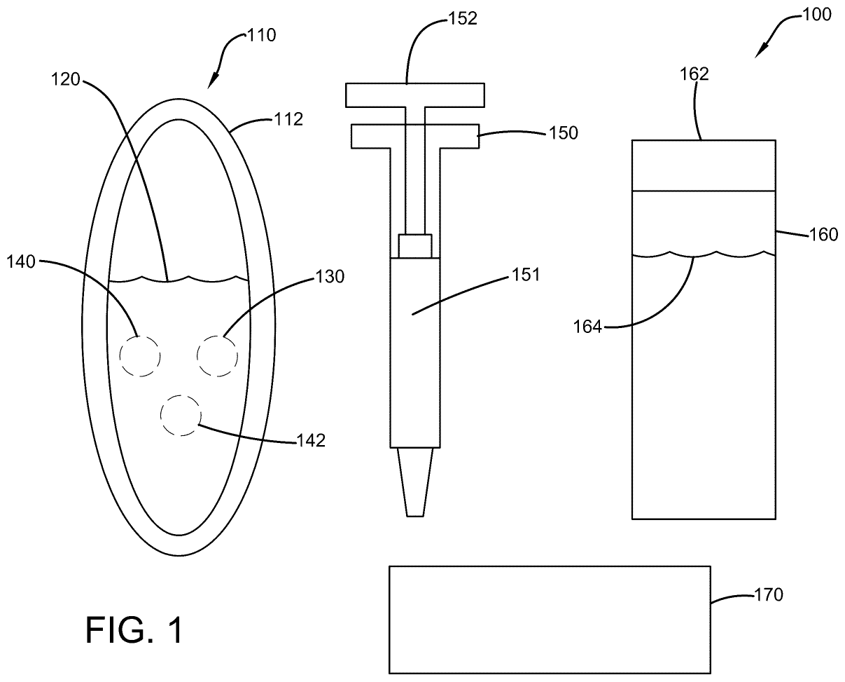

Referring initially to the drawings, FIG. 1 illustrates a perspective view of one potential embodiment of a time-release vaginal lubrication capsule device 100 of the present invention in accordance with the disclosed architecture. The device 100 is comprised of a capsule 110, which can take on any capsule shape, such as an elongated oval, sphere, or other appropriate design suited for ease of insertion and comfort. During use, the capsule 110 is inserted into a user's vagina, where it reacts to the body's natural heat. More specifically, the capsule 110 is comprised of a biocompatible, heat-sensitive, and dissolvable polymer. This polymer is designed to break down at a controlled, gradual rate as it reaches normal internal body temperature (approximately 37° C./98.6° F.). Upon reaching this threshold, the polymer becomes unstable and begins to dissolve in a controlled manner, allowing the release of a lubricant 120 stored within the capsule 110. This ensures that the lubricant is released slowly and steadily into the vaginal canal over a designated period of time, providing sustained moisture and comfort during intimate activities.

More specifically, the capsule 110 is preferably composed of biodegradable polymers such as, but not limited to, poly (lactic-co-glycolic acid) (PLGA), a material commonly used in medical implants and drug delivery systems due to its biocompatibility and predictable degradation profile. The PLGA polymer is chosen for its ability to degrade at body temperature and moisture levels found in the vaginal environment. The breakdown of the polymer is designed to be safe and non-irritating to sensitive tissues, ensuring that the user experiences minimal discomfort during the dissolution process. In some embodiments, the dissolution process is further aided by the presence of moisture, such as natural vaginal fluids, which help to accelerate the breakdown of the polymer.

The rate at which the capsule 110 dissolves can be adjusted depending on the desired time-release duration. For example, in embodiments where a longer release period is needed, the capsule wall 112 may be made thicker. A thicker wall 112 slows the dissolution process, delaying the full release of the lubricant 120. Conversely, for shorter time intervals, a thinner wall 112 may be used, allowing quicker breakdown and more rapid release of the lubricant.

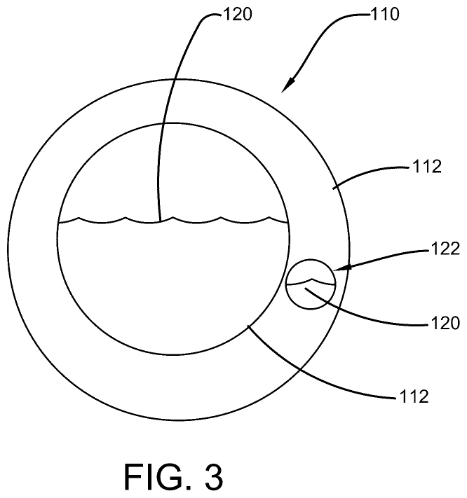

In another embodiment, the capsule 110 may be constructed with a multi-layer design, as seen in FIG. 3. The capsule 110 would feature a plurality of wall layers 112 that dissolve sequentially. Lubricant 120 is positioned between each layer, ensuring that with the dissolution of each layer, a fresh batch of lubricant is released. This tiered release system allows the device to dispense lubricant at predetermined intervals, ensuring ongoing moisture and lubrication for the user over an extended period. This design ensures that lubrication is steadily delivered in phases rather than all at once, providing long-lasting comfort without reapplication.

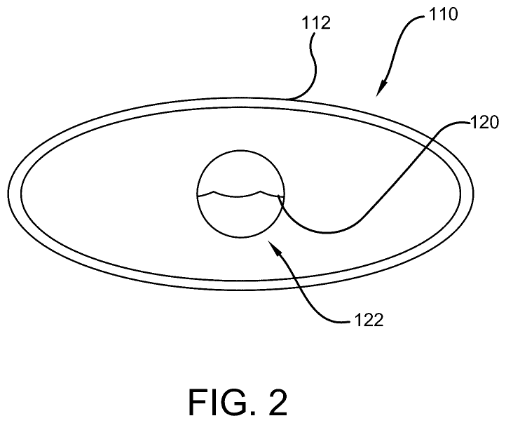

In one embodiment, the lubricant 120 contained within the capsule 110 is in liquid form, designed to be quickly absorbed by the vaginal tissue for immediate relief from dryness. In another embodiment, the lubricant 120 may be micro-encapsulated in beads 122, as seen in FIG. 2 and FIG. 3. The beads 122, like the capsule 110 itself, are made from a dissolvable material, often similar or identical to the biodegradable polymer used in the capsule. The beads 122 dissolve over time at varying rates, creating a staggered release of the lubricant 120. This method is particularly useful for ensuring a slow and steady release of lubricant over an extended period. In one embodiment, a plurality of beads 122 may be incorporated, each bead potentially made from a different material or having different wall thicknesses. The variation in material composition and thickness allows for the beads 122 to dissolve at different rates, ensuring continuous and extended lubrication as each set of beads breaks down at its specific time interval.

The lubricant 120 itself is preferably water-based, which allows for easy absorption and reduces the risk of irritation or allergic reactions compared to oil-based products. The lubricant 120 may also contain soothing agents 142 such as hyaluronic acid and/or aloe vera to promote healing and hydration, particularly for users who experience discomfort due to dryness or irritation. These agents 142 are known for their ability to enhance moisture retention and promote healthy skin or tissue recovery, making the lubricant 120 more than just a short-term solution for dryness, but also a tool for improving overall vaginal health.

In some embodiments, the lubricant 120 may also be flavored 130 and/or scented 140 to enhance the user experience. The flavors 130 may include any food-safe flavor, such as but not limited to a strawberry, vanilla, or mint, while the scents 140 may range from natural, hypoallergenic options like lavender or rose. The flavor 130 and scent 140 options are designed to be hypoallergenic, ensuring that they do not irritate sensitive areas, while also providing a customizable experience for the user.

In one embodiment, the device 100 is comprised of at least one syringe 150, wherein the capsule 110 can be placed within an interior space 151 of the syringe 150 and insert into a vagina via pressing down at least one plunger 152 of the syringe 150. The device 100 may also be comprised of a sterilization cylinder 160 that can receive the capsule 110. The cylinder 160 is comprised of at least one lid 162 that can be closed once the capsule 110 and/or syringe 150 are inserted within, along with a cleaning solution 164 to clean/sterilize the same. The capsule 110, syringe 150, and cylinder 160 may be stored in at least one case 170 of any style and configuration.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “time-release vaginal lubrication capsule device” and “device” are interchangeable and refer to the time-release vaginal lubrication capsule device 100 of the present invention.

Notwithstanding the forgoing, the time-release vaginal lubrication capsule device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the time-release vaginal lubrication capsule device 100 as shown in the FIGS. are for illustrative purposes only, and that many other sizes and shapes of the time-release vaginal lubrication capsule device 100 are well within the scope of the present disclosure. Although the dimensions of the time-release vaginal lubrication capsule device 100 are important design parameters for user convenience, the time-release vaginal lubrication capsule device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A time-release vaginal lubrication capsule device comprising:
a capsule configured for insertion into a vagina, the capsule being comprised of a biocompatible, a heat-sensitive, and a dissolvable polymer, wherein the polymer is designed to dissolve at body temperature; and
a lubricant stored within the capsule.

2. The time-release vaginal lubrication capsule device of claim 1, wherein the biocompatible, a heat-sensitive, and a dissolvable polymer is comprised of a PLGA polymer.

3. The time-release vaginal lubrication capsule device of claim 1, wherein the lubricant is comprised of a water-based lubricant.

4. The time-release vaginal lubrication capsule device of claim 1, wherein the lubricant is comprised of a soothing agent.

5. The time-release vaginal lubrication capsule device of claim 4, wherein the soothing agent is comprised of an aloe vera.

6. The time-release vaginal lubrication capsule device of claim 4, wherein the soothing agent is comprised of a hyaluronic acid.

7. The time-release vaginal lubrication capsule device of claim 1, wherein the lubricant is comprised of a flavored lubricant.

8. The time-release vaginal lubrication capsule device of claim 4, wherein the soothing agent is comprised of a scented lubricant.

9. The time-release vaginal lubrication capsule device of claim 1 further comprised of a syringe comprised of a plunger.

10. A time-release vaginal lubrication capsule device comprising:
a capsule having a wall composed of a biodegradable polymer, wherein the wall dissolves at a controlled rate upon exposure to a heat; and
a lubricant positioned within the capsule, wherein the is released gradually as the wall dissolves.

11. The time-release vaginal lubrication capsule device of claim 10, wherein the lubricant is positioned within a bead.

12. The time-release vaginal lubrication capsule device of claim 11, wherein the bead is comprised of a biodegradable polymer.

13. A time-release vaginal lubrication capsule device comprising:
a capsule comprised of a first wall and a second wall, wherein the first wall and the second wall dissolve when exposed to a body temperature; and
a lubricant positioned between the first wall and the second wall.

14. The time-release vaginal lubrication capsule device of claim 13, wherein the first wall is thicker than the second wall.

15. The time-release vaginal lubrication capsule device of claim 13, wherein the lubricant is contained within a bead.

16. The time-release vaginal lubrication capsule device of claim 15, wherein the bead is comprised of a biodegradable polymer.

17. The time-release vaginal lubrication capsule device of claim 13, wherein the lubricant is comprised of a water-based lubricant.

18. The time-release vaginal lubrication capsule device of claim 17, wherein the lubricant is comprised of a hypoallergic flavor.

19. The time-release vaginal lubrication capsule device of claim 17, wherein the lubricant is comprised of a hypoallergic scent.

20. The time-release vaginal lubrication capsule device of claim 13 further comprised of a sterilization cylinder comprised of a lid.
